(19) United States
(12) Patent Application Publication
(10) Pub. No.: US 2025/0295574 A1
(43) Pub. Date: Sep. 25, 2025

(54) Skin Balm Composition and Associated Methods

(71) Applicant: Molly Thompson, Hot Springs Village, AR (US)

(72) Inventor: Molly Thompson, Hot Springs Village, AR (US)

(21) Appl. No.: 19/216,176

(22) Filed: May 22, 2025

Related U.S. Application Data
(60) Provisional application No. 63/568,530, filed on Mar. 22, 2024.

(51) Int. Cl. A61K 8/92; A61K 8/9789; A61K 8/9794; A61Q 19/00

(52) U.S. Cl. CPC A61K 8/922; A61K 8/927; A61K 8/9789; A61K 8/9794; A61Q 19/007; A61K 2236/37

(57) ABSTRACT

Provided is a skin balm application device including: an applicator; a skin balm composition including ingredients of: shea butter; cocoa butter; beeswax; soybean oil; sunflower oil; aloe; and borage oil; and a cap. Provided also is a method, including: (a) double boil shea butter, cocoa butter, and beeswax; (b) add soybean oil, sunflower oil, aloe, and borage oil; (c) mix all the above ingredients together; and (d) pour the mixture into containers.

### CROSS REFERENCE TO RELATED APPLICATIONS

This application claims the benefit of U.S. Provisional Application Ser. No. 63/568,530 filed Mar. 22, 2024, and entitled “SKIN BALM COMPOSITION TO HELP PSORIASIS AND HERPES, AS WELL AS ITCHING AND BURNS” the entirety of which is herein incorporated by reference.

### TECHNICAL FIELD

The present disclosure generally relates to creams and balms, specifically creams and balms to treat skin conditions such as cold sores, burns, bug bites and itching.

### BACKGROUND

Currently, balms and creams are used to treat skin conditions such as psoriasis, but have not been shown to be effective, due to taking too much time, not having that much impact in terms of healing, and not preventing further outbreaks. Also some balms and creams only treat psoriasis, but not other conditions such as cold sores, burns, bug bites and itching.

As a result, there is a need in the art for a skin balm composition that effectively treats psoriasis that is also better than most of the prescription medications out there.

### SUMMARY

Provided is a skin balm composition that effectively treats psoriasis and herpes, as well as itching and burns. Said skin composition utilizes natural ingredients, including essential oils and aloe.

Other objects and advantages of the present disclosure will become obvious to the reader and it is intended that these objects and advantages are within the scope of the present disclosure. To the accomplishment of the above and related objects, this disclosure may be embodied in the form illustrated in the accompanying drawings, attention being called to the fact, however, that the drawings are illustrative only, and that changes may be made in the specific construction illustrated and described within the scope of this application.

This has outlined, rather broadly, the features and technical advantages of the present disclosure in order that the detailed description that follows may be better understood. Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following description taken in conjunction with the accompanying drawings.

FIG. 1 is a view illustrating a skin balm composition contained within an applicator according to an aspect of the present disclosure.

FIG. 2 is a view illustrating another skin balm composition contained within an applicator according to an aspect of the present disclosure.

FIG. 3 is a view illustrating another skin balm composition contained within an applicator according to an aspect of the present disclosure.

Example embodiments will become more fully understood from the detailed description given herein below and the accompanying drawings, wherein like elements are represented by like reference characters, which are given by way of illustration only and thus are not limitative of the example embodiments herein.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts. As described herein, the use of the term “and/or” is intended to represent an “inclusive OR”, and the use of the term “or” is intended to represent an “exclusive OR”.

Provided is a skin balm application device including: an applicator; a skin balm composition including ingredients of: shea butter; cocoa butter; beeswax; soybean oil; sunflower oil; aloe; and borage oil; and a cap. Provided also is a method, including: (a) double boil shea butter, cocoa butter, and beeswax; (b) add soybean oil, sunflower oil, aloe, and borage oil; (c) mix all the above ingredients together; and (d) pour the mixture into containers.

As stated above, in a preferred embodiment, the present invention is directed towards a skin balm composition that has been shown to be more effective than prescription psoriasis medication. In a preferred embodiment, the skin balm composition of the present invention comprises natural, gentle ingredients only including essential oils and aloe. Those natural ingredients also include shea butter, cocoa butter, beeswax, soybean oil, sunflower oil, aloe and borage oil. These natural ingredients were chosen because they have healing properties for the skin and are gentle for sensitive skin. The skin balm composition of the present invention has been shown to help alleviate psoriasis outbreaks and has been shown to eliminate cold sores, while also helping with burns, bug bites, and itching as well.

The claimed invention differs from what currently exists. The skin balm composition of the present invention is better than anything in the field because it uses only primary ingredients that are all natural. Results have shown to be highly effective.

Conventional psoriasis medications are very harsh on the body. In addition, conventional balms can take a long time to work. In contrast, the skin balm composition of the present invention has been shown to be effective within 24-48 hours of application.

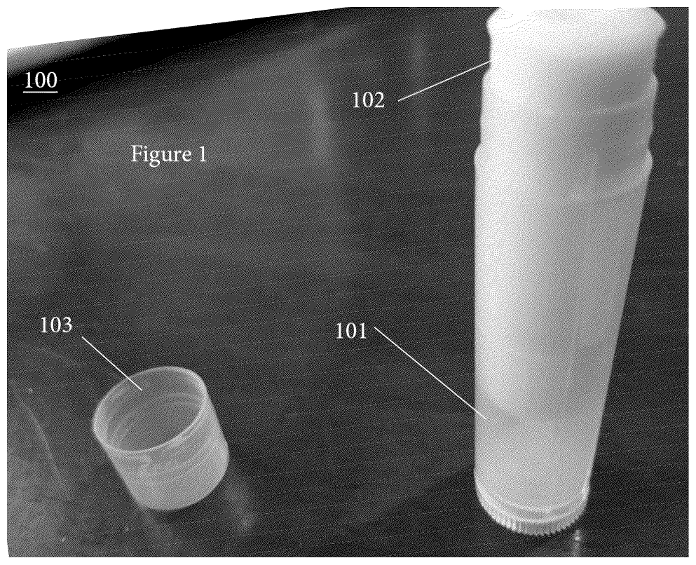

As described herein, and as seen in FIGS. 1-3 hereto, in a preferred embodiment the skin balm composition of the present invention comprises the following primary components.
1. Double boil shea butter, cocoa butter, and beeswax.
2. Add soybean oil, sunflower oil, aloe, and borage oil.
3. Mix and pour into containers

Once the balm is made and cooled, it can be applied liberally as needed.

### How to Make the Invention:

Mix the ingredients in the steps provided.
All elements are necessary to function properly.
In a preferred embodiment, the components are added in equal parts.

### How to Use the Invention:

Apply to the skin liberally as needed.

According to FIG. 1, an applicator 101 is shown, storing skin balm composition 102, and there is also a cap 103. The skin balm composition 102 can be formed into the shape of a mold and be fit into the applicator 101. The skin balm composition 102 can take up the entire applicator 101 or only take up a portion of the applicator 101 as well. The top exposed portion of the skin balm composition 102 can be applied to the skin of the human user or consumer, and the means of applying it can be liberal as well. In one aspect, the cap 103 can be placed over the skin balm composition 102 to cover it. This leads to enhanced preservation of the skin balm composition 102, protection from weather and also prevents it from drying out or being overly exposed to the air or atmosphere. The applicator 101 can also be made out of materials including plastic, silicone, stainless steel, or thermoplastic elastic (TPE).

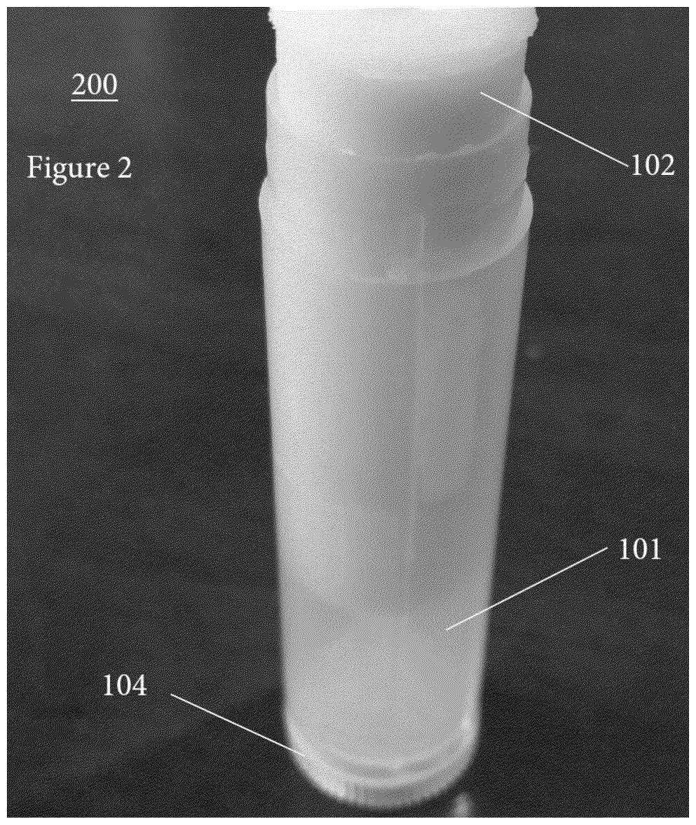

According to FIG. 2, the applicator 101 is also shown with the skin balm composition 102 being pushed slightly outside of. According to one aspect, the applicator 101 may have a twist mechanism 104 at the bottom of the applicator 101 that allows a human user to turn in order to extend and push out more of the skin balm composition 102 to apply the skin balm composition 102 liberally onto the skin of the human user.

According to one aspect, the applicator 101 has a push mechanism that allows a human user to push out the skin balm composition 102 more in order to apply liberally to the skin of the human user—this push mechanism is not shown in FIG. 2 but would be located at the bottom of the applicator 101, where the twist mechanism 104 currently is shown in FIG. 2.

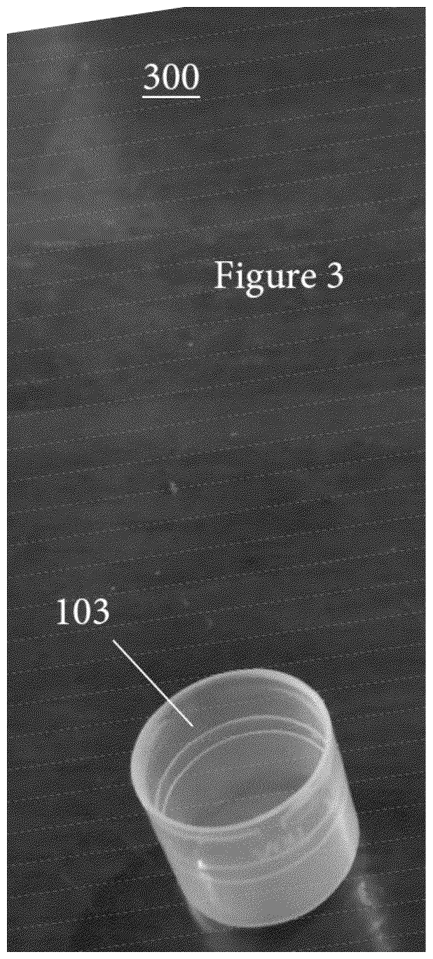

According to FIG. 3, the cap 103 is shown. The cap 103 can be placed on top of the applicator 101 over the skin balm composition 102 to cover it. This leads to enhanced preservation of the skin balm composition 102, protection from weather and also prevents the skin balm composition 102 from drying out or being overly exposed to the air or atmosphere. The cap 103 can also be made out of materials including plastic, silicone, stainless steel, or thermoplastic elastic (TPE).

According to an aspect of the disclosure, the applicator 101 can be ajar instead, the skin balm composition can be stored in the jar, and the cap 103 becomes ajar lid, so when closed or sealed, the skin balm composition can be sealed.

According to an aspect of the disclosure, there can be a motor or electronics that drive the applicator, the applicator push mechanism and/or the applicator twist mechanism.

According to one aspect of the disclosure, any of the above-described components can be equipped with electronics, micro-electronics, chips, or other similar electrical components. Take for example the applicator. The applicator can be made from such electronic components and utilize the processors described below.

A or several processors may be utilized in connection with various apparatuses and methods. These processors may be implemented using electronic hardware, computer software, or any combination thereof. Whether such processors are implemented as hardware or software will depend upon the particular application and overall design constraints imposed on the system. By way of example, a processor, any portion of a processor, or any combination of processors presented in this disclosure may be implemented with a microprocessor, microcontroller, digital signal processor (DSP), a field-programmable gate array (FPGA), a programmable logic device (PLD), a state machine, gated logic, discrete hardware circuits, and other suitable processing components configured to perform the various functions described throughout this disclosure. The functionality of a processor, any portion of a processor, or any combination of processors presented in this disclosure may be implemented with software being executed by a microprocessor, microcontroller, DSP, or other suitable platform.

Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software may reside on a computer-readable medium. A computer-readable medium may include, by way of example, memory such as a magnetic storage device (e.g., hard disk, floppy disk, magnetic strip), an optical disk (e.g., compact disc (CD), digital versatile disc (DVD)), a smart card, a flash memory device (e.g., card, stick, key drive), random access memory (RAM), read only memory (ROM), programmable ROM (PROM), erasable PROM (EPROM), electrically erasable PROM (EEPROM), a register, or a removable disk. Although memory is shown separate from the processors in the various aspects presented throughout this disclosure, the memory may be internal to the processors (e.g., cache or register).

Computer-readable media may be embodied in a computer-program product. By way of example, a computer-program product may include a computer-readable medium in packaging materials. Those skilled in the art will recognize how best to implement the described functionality presented throughout this disclosure depending on the particular application and the overall design constraints imposed on the overall system.

It is to be understood that the specific order or hierarchy of steps in the methods disclosed is an illustration of exemplary processes. Based upon design preferences, it is understood that the specific order or hierarchy of steps in the methods may be rearranged. The accompanying method claims present elements of the various steps in a sample order, and are not meant to be limited to the specific order or hierarchy presented unless specifically recited therein.

For a firmware and/or software implementation, the methodologies may be implemented with modules (e.g., procedures, functions, and so on) that perform the functions described herein. A machine-readable medium tangibly embodying instructions may be used in implementing the methodologies described herein. For example, software codes may be stored in a memory and executed by a processor unit. Memory may be implemented within the processor unit or external to the processor unit. As used herein, the term “memory” refers to types of long term, short term, volatile, nonvolatile, or other memory and is not to be limited to a particular type of memory or number of memories, or type of media upon which memory is stored.

If implemented in firmware and/or software, the functions may be stored as one or more instructions or code on a computer-readable medium. Examples include computer-readable media encoded with a data structure and computer-readable media encoded with a computer program. Computer-readable media includes physical computer storage media. A storage medium may be an available medium that can be accessed by a computer. By way of example, and not limitation, such computer-readable media can include RAM, ROM, EEPROM, CD-ROM or other optical disk storage, magnetic disk storage or other magnetic storage devices, or other medium that can be used to store desired program code in the form of instructions or data structures and that can be accessed by a computer; disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

Although the present disclosure and its advantages have been described in detail, it should be understood that various changes, substitutions and alterations can be made herein without departing from the technology of the disclosure as defined by the appended claims. For example, relational terms, such as “above” and “below” are used with respect to a substrate or electronic device. Of course, if the substrate or electronic device is inverted, above becomes below, and vice versa. Additionally, if oriented sideways, above and below may refer to sides of a substrate or electronic device. Moreover, the scope of the present application is not intended to be limited to the particular configurations of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed

1. A skin balm application device comprising:
an applicator;
a skin balm composition comprising ingredients of:
shea butter;
cocoa butter;
beeswax;
soybean oil;
sunflower oil;
aloe; and
borage oil; and
a cap.

2. The skin balm application device of claim 1, wherein the applicator is made of ingredients comprising plastic, silicone, stainless steel, or thermoplastic elastic (TPE).

3. The skin balm application device of claim 1, wherein the cap is made of ingredients comprising plastic, silicone, stainless steel, or thermoplastic elastic (TPE).

4. The skin balm application device of claim 1, wherein the applicator has a twist mechanism that allows more pushing out of the skin balm composition for liberal application upon skin of a human user.

5. The skin balm application device of claim 1, wherein the applicator has a push mechanism that allows more pushing out of the skin balm composition for liberal application upon skin of a human user.

6. The skin balm application device of claim 1, wherein the applicator is a jar.

7. The skin balm application device of claim 6, wherein the skin balm composition is stored within the jar.

8. The skin balm application device of claim 6, wherein the cap is a jar lid that can be screwed tightly to secure the skin balm composition within the jar.

9. The skin balm application device of claim 4, wherein the twist mechanism can be driven by a motor or electronics.

10. The skin balm application device of claim 5, wherein the push mechanism can be driven by a motor or electronics.

11. The skin balm application device of claim 1, wherein the ingredients are added all in equal parts.

12. A method, comprising:
(a) double boil shea butter, cocoa butter, and beeswax;
(b) add soybean oil, sunflower oil, aloe, and borage oil;
(c) mix all the above ingredients together; and
(d) pour the mixture into containers.

13. The method of claim 12, further comprising:
(e) adding all the ingredients in equal parts.

14. The method of claim 12, further comprising:
(e) pour the mixture from containers into an applicator;
(f) using a mechanism to extend the mixture from the applicator; and
(g) applying the mixture liberally to the skin of a human user.

15. The method of claim 14, wherein the mechanism comprises a twist mechanism used to extend the mixture from the applicator.

16. The method of claim 16, wherein the twist mechanism is driven by a motor or electronics.

17. The method of claim 14, wherein the mechanism comprises a push mechanism used to extend the mixture from the applicator.

18. The method of claim 16, wherein the twist mechanism is driven by a motor or electronics.

19. The method of claim 12, further comprising:
(e) pour the mixture from containers into a jar;
(f) pouring the mixture from the jar to apply liberally to the skin of a human user.

20. The method of claim 17, further comprising:
(g) securing a lid on the top of the jar to preserve the skin balm composition.
